(19) United States
(12) Patent Application Publication
(10) Pub. No.: US 2016/0058945 A1
(43) Pub. Date: Mar. 3, 2016

(54) Injection Device with Audio Output

(71) Applicant: Thomas A. Piscitelli, Williamsburg, VA (US)

(72) Inventor: Thomas A. Piscitelli, Williamsburg, VA (US)

(21) Appl. No.: 14/472,077

(22) Filed: Aug. 28, 2014

(51) Int. Cl. A61M 5/20; A61M 5/168; A61M 5/172; A61M 5/32

(52) U.S. Cl. CPC A61M 5/20; A61M 5/3243; A61M 5/1684; A61M 5/172; A61M 2005/2026; A61M 2205/507; A61M 2205/50; A61M 2205/52; A61M 2205/581; A61M 2205/583; A61M 2205/3584; A61M 2205/3592

(57) ABSTRACT

A medical injection device and associated methods of use are disclosed which facilitate the self-administration of injectable medicine by a user who is visually impaired. In an aspect, the medical injection device and associated methods assist a visually impaired user by providing a speaker attached to the injection device that communicates audibly with a user the size of the dosage of medicine the device is currently set to administer, as well as what amount of medicine is currently available within the device.

### FIELD OF THE DISCLOSURE

The present disclosure generally relates to medical devices and more particular to devices for injecting medication that include audio output functionality.

### BACKGROUND

The statements in this section merely provide background information related to the present disclosure and may not constitute prior art.

Devices for injecting an individual with medication are well known in the art. Particularly, insulin pens for delivering adjustable specified dosages of insulin to user patients have been around for some time. The popularity of such pens is due do the convenience they offer to users with regards to transporting and administering insulin dosages.

There are two main types of conventional insulin pens currently on the market: those that may be disposed of after administering a single insulin dosage, and those that use removable disposable insulin cartridges that may serve to administer many dosages. Regardless of type, an insulin pen has three general parts: a base that contains a dosage measuring mechanism, a holder for an insulin cartridge, and a removable cap that may cover the injection needle.

Regardless of the type of pen used, current insulin pens on the market rely on a visual display to communicate to users the dosage of insulin to be administered. Due to the inherently small size of the pens, the dosage display is necessarily small. This can be problematic, especially for diabetic patients who are primarily the ones who need insulin, because one complication of diabetes is reduced visual acuity. A user who is not able to see the dosage on an insulin pen becomes dependent on others for help in administering the medication, or must seek that aid of visual enhancement mechanisms, such as magnifying lenses, which takes away from the convenience offered by the pens in the first place.

Attempts have been made to make insulin pens more user-friendly for the visually impaired. For example, magnifiers have been made available that fit onto some pens. However, a user is still required to rely completely on a visual determination to set the correct dosage. Some pens have been developed that make clicking noises to help a user “hear” the dosage being set. However, clicks may be hard to distinguish from background noises, and thus lack a significant level of certainty with regard to the dosage actually being administered. Uncertainty is generally very unwelcome an anything medical related, particularly regarding medicine dosage amounts.

Given the foregoing, devices and associated methods are needed that allow visually impaired users to be able to self-administer medical injections. More particularly, insulin pens that provide a clear means to determine dosage other than by visual displays or mere clicking sounds are required.

### SUMMARY

This Summary is provided to introduce a selection of concepts. These concepts are further described below in the Detailed Description section. This Summary is not intended to identify key features or essential features of this disclosure's subject matter, nor is this Summary intended as an aid in determining the scope of the disclosed subject matter.

Aspects of the present disclosure meet the above-identified needs by providing devices and associated methods which facilitate the self-administering of medication, particularly insulin, by users without having to rely on visual or uncertain auditory cues. Specifically, in an aspect, devices and associated methods are disclosed wherein such devices provide auditory output in the form of a verbal communication via a speaker that communicates to a user the dosage of insulin about to be administered.

In an aspect, the disclosed device comprises a controlling electronics. Such controlling electronics may perform computational functions such as receiving input commands and generating appropriate outputs. Furthermore, such a controlling electronics may perform basic calculation and measurement functions, such as, by way of example and not limitation, determining how many doses of medicine are left in the device. Such functions may be accomplished by means of at least one microchip or other similar device(s) as recognized by those skilled in the relevant art(s) after reading the description herein.

In some aspects, the controlling electronics further comprise a form of computer-based memory. Such memory may be useful for, by way of example and not limitation, keeping track of how many doses have been administered from a current insulin cartridge, as well as the size of such doses.

In some aspects, the verbal communication utilized by the disclosed device comprises pre-recorded human readings of possible dosages and other phrases to be used. In other aspects, the verbal communication comprises computer-generated words.

In some aspects, a user presses a button communicatively coupled to the controlling electronics in order to receive a communication of what dose the device is currently set to administer and presses a different but similarly configured button to receive a communication of how many dosages are left in the device. In other aspects, the speaker is wired such as to also function as a microphone. In these aspects, a user may speak into the microphone to request such dosage information.

In some aspects, a user may speak pre-determined phrases into the microphone in order to obtain the desired information. By way of example and not limitation, such a phrase may be, “Doses remaining.” Upon input of such a phrase, the controlling electronics within the device may communicate the dosages left in the device via the speaker. In other aspects, the controlling electronics may be configured to interpret a various number of free-form phrases spoken by the user. If a particular phrase is unintelligible by the controlling electronics, a verbal communication may be made to the user stating such. By way of example and not limitation, such a verbal communication may be “Please restate your command.”

In some aspects, the disclosed device may be programmed to communicate with users via different languages.

Further features and advantages of the present disclosure, as well as the structure and operation of various aspects of the present disclosure, are described in detail below with reference to the accompanying drawings.

### BRIEF DESCRIPTION OF THE DRAWINGS

The features and advantages of the present disclosure will become more apparent from the Detailed Description set forth below when taken in conjunction with the drawings in which like reference numbers indicate identical or functionally similar elements.

FIG. 1 is a cross-sectional view of an injection device for generating audio communications of medicinal dosages, according to an aspect of the present disclosure.

FIG. 2 is a front view of an injection device for generating audio communications of medicinal dosages, according to an aspect of the present disclosure.

FIG. 3 is a flowchart illustrating an exemplary process for injecting a specific dose of medication using an injection device for generating audio communications of medicinal dosages, according to an aspect of the present disclosure.

FIG. 4 is a flowchart illustrating an exemplary process for facilitating the determination of how much medicine is remaining in an injection device for generating audio communications of medicinal dosages, according to an aspect of the present disclosure.

FIG. 5 a block diagram of an exemplary computer system useful for implementing various aspects of the present disclosure.

### DETAILED DESCRIPTION

The present disclosure is directed to devices and associated methods that facilitate the self-administration of medicine, particularly insulin, via injection means by providing a clear, verbal audio indication of what dosage is currently set to be administered, as well as how many dosages are remaining in the given device.

The term “injection device” and/or the plural form of this term are used throughout herein to refer to any mechanism used to inject an individual with a medication.

The term “user” and/or the plural form of this term are used throughout herein to refer to any medical patient who may receive medication via injection of such medication into a part of the body.

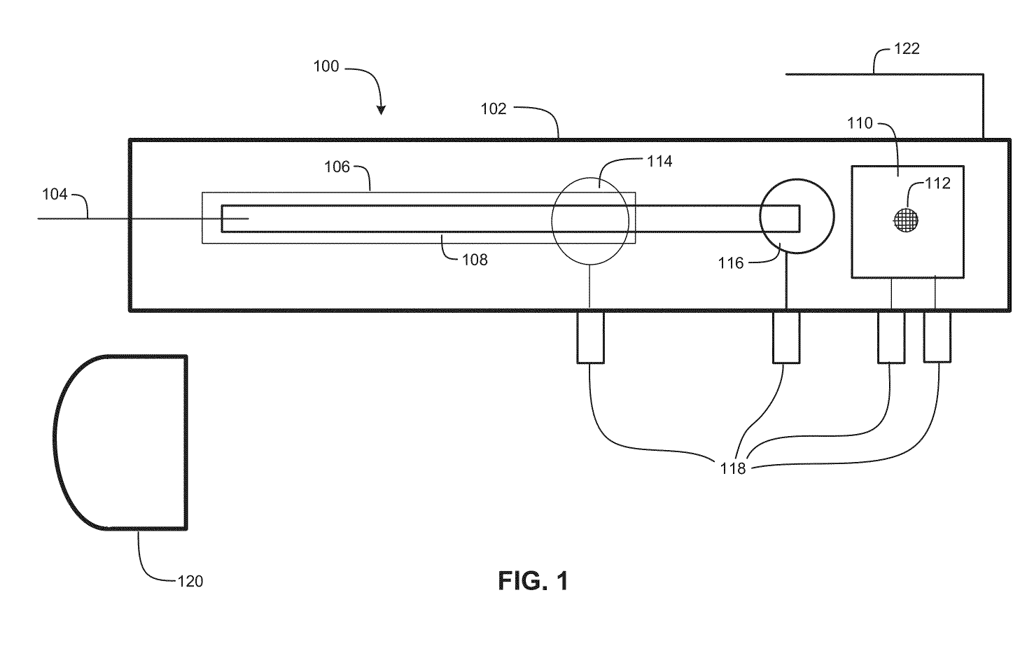

Referring now to FIG. 1, a cross-sectional view of an injection device 100 for generating audio communications of medicinal dosages, according to an aspect of the present disclosure, is shown.

As shown in FIG. 1, injection device 100 comprises a body 102, a needle 104, a cartridge holder 106, a removable cartridge 108, controlling electronics 110, a speaker 112, a needle retractor 114, a dispense mechanism 116, buttons 118, a cap 120, and a belt clip 122.

In an aspect, controlling electronics 110 comprises a microchip and/or one or more other computing instruments such as would be recognized by those skilled in the relevant art(s) after reading the description herein as being able to perform computing functions within the size allotted by device 100. Such computing functions may include receiving inputs and generating outputs, performing calculations, and making determinations. In some aspects, controlling electronics 110 further comprises devices that are capable of performing data storage functions, such as memory chips and similar items as recognized by those skilled in the relevant art(s).

In some aspects, two of buttons 118 are communicatively coupled to controlling electronics 110, either by wire or wirelessly. Controlling electronics 110 are further connected to speaker 112. Depressing one of buttons 118 may cause controlling electronics 110 to make a determination of what dosage device 100 is currently set to administer. Upon such a determination, controlling electronics 110 may send an output to a user via speaker 112 in the form of a verbal audio communication as to such. Such verbal audio communication may be in the form of a pre-recorded human voice, a computer-generated voice, or any similar form as recognized by those skilled in the relevant art(s). Repeatedly pressing button 118 may cause the set dosage amount to change incrementally. In a similar fashion, depressing the other button 118 may cause, via a substantially similar process, controlling electronics 110 to make a determination as to what amount of medication are left in device 100 and communicate such a determination to a user.

In some aspects, speaker 112 is a micro speaker mounted externally on the upper portion of device 100 and is communicatively coupled to controlling electronics 110, either by wires or wirelessly. In other aspects, speaker 112 is of any size, design, and configuration as would be recognized as appropriate by those skilled in the relevant art(s).

In some aspects, speaker 112 may be configured with controlling electronics 110 such as to also function as a microphone. In such aspects, a user may speak a verbal command into speaker 112 in order to receive the desired outputs. In some aspects, such commands may be pre-determined commands that elicit specific responses from controlling electronics 110. In other aspects, the commands may be of a free-form nature that may be interpreted by controlling electronics 110 in order to formulate an appropriate response thereto. By way of example and not limitation, a pre-determined command may comprise the phrase, “Dosages remaining.” Upon receipt of such a phrase, controlling electronics 110 may determine and then communicate to a user how many doses are left within device 100. In some aspects, a user presses a button and then speaks into speaker 112. In other aspects, a user holds down the button the entire time while speaking into speaker 112. If a user phrase is not understood by controlling electronics 110, a response indicating such may be provided to the user. By way of example and not limitation, such a response may comprise the phrase, “Please restate your command.”

In an aspect, injection device 100 includes a button 118 communicatively connected to dispense mechanism 116. Such a connection may be either physical or electronic. Upon the depression of button 118, dispense mechanism 116 may inject an amount of medicine from cartridge 108 equal to the dosage that device 100 is set to administer, as determined by controlling electronics 110. Such injection may occur via a connection to cartridge 108 and may be by any means as recognized by those skilled in the relevant art(s) after reading the description herein.

In some aspects, injection device 100 comprises a button 118 connected to cartridge holder 106. Such a connection may be either physical or electronic. Cartridge holder 106 may function to hold cartridge 108 in place as well as to connect it to needle 104. Upon depression of button 118, needle retractor 114 may be engaged such as to move needle 104 between a position completely within body 102 and an exposed position protruding from the lower end of body 102 such that it may be ready for use. Alternatively, a sliding mechanism, rotating mechanism, or other similar mechanism as will be appreciated by those skilled in the relevant art(s) after reading the description herein may engage needle retractor 114 instead of button 118. In other aspects, device 100 does not have a retractable needle. Instead, cap 120 may be used to cover needle 104 when it is not in use. In still other aspects, needle 104 may be removed from body 102 when it is not in use. In such aspects when needle 104 is removed from body 102, needle 104 may or may not be further attached to cartridge 108 and removed therewith. When needle 104 is not attached to cartridge 108, the lower end of body 102 may comprise an interface for securely receiving needle 104 and connecting it to cartridge 108. Such an interface may comprise clips, snaps, or similar mechanisms as recognized by those skilled in the relevant art, with corresponding mechanisms on needle 104. A removed needle 104 and/or removed cartridge 108 may be stored for later use or may be disposed of and replaced in order to promote safety and sanitization. Such disposal of needle 104 may occur after each use, or, especially in the aspects in which needle 104 is attached to cartridge 108, disposal may occur when cartridge 108 does not have any dosages remaining. Cap 120 may be used with any configuration of needle 104 such as to provide extra safety and security to device 100.

In some aspects, device 100 comprises belt clip 122 to facilitate the transportation of such device 100 via attachment to a user's garment.

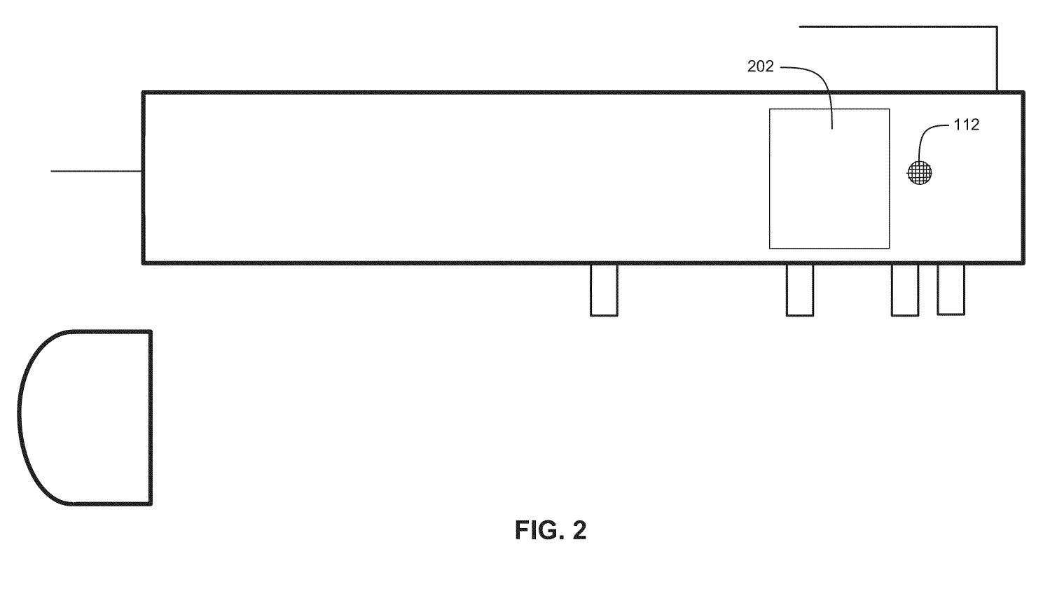

Referring now to FIG. 2, a front view of injection device 100 for generating audio communications of medicinal dosages, according to an aspect of the present disclosure, is shown.

In some aspects, injection device 100 comprises display screen 202. Display screen 202 may comprise an LCD type display, or other similar visual output as recognized by those skilled in the relevant art(s). Such a display may be useful in receiving visual confirmation of the audio output communication received from device 100. Additionally, screen 202 may be used by a user instead of the audio outputs for situations in which audio noises would not be appropriate. In other aspects, display screen 202 may comprise any means of visually showing the current set dosage of device 100 and the amount of medicine remaining within cartridge 108 as recognized by those skilled in the relevant art(s) after reading the description herein.

In some aspects, display screen 202 may include a user interface that allows a user to input commands. Such a user interface may comprise a touchscreen, physical buttons, or any other similar configuration as would be recognized by those skilled in the relevant art(s) after reading the description herein.

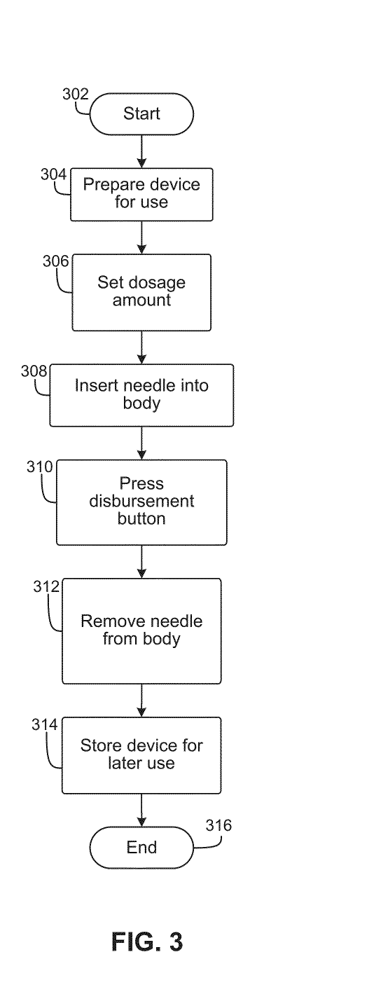

Referring now to FIG. 3, a flowchart illustrating an exemplary process 300 for injecting a specific dose of medication using injection device 100 for generating audio communications of medicinal dosages, according to an aspect of the present disclosure, is shown.

Process 300, which may be executing utilizing device 100, begins at step 302 with control passing immediately to step 304.

At step 304, a user prepares injection device 100 for use. In some aspects, this is done by removing cap 122. In other aspects, there is no cap 122. Once cap 122 is removed, needle 104 may be exposed. In some aspects, a button 118 or other mechanism may need to be engaged to expose needle 104 by deploying it from body 102. In other aspects, needle 104 may need to be attached to device 100 at the lower portion of body 102. In still other aspects, a cartridge 108 may be attached to needle 104 and require insertion into the body 102 of device 100 as needle 104 is attached.

At step 306, user presses a different button 118 communicatively coupled to controlling electronics 110 to set the dosage for the injection. In some aspects, user keeps pressing button 118 to toggle through the possible dosages as they are communicated from controlling electronics 110 through speaker 112. User then stops when the correct dosage is heard. In other aspects, user presses 118 and verbally speaks a desired dosage. In still other aspects, user uses display screen 202 to enter the desired dosage. Once the desired dosage is set, controlling electronics 110 send a signal to dispense mechanism 116 indicating how much medicine to dispense upon engagement of mechanism 116. In an aspect, dispense mechanism 116 may contain a microchip or other similar instrument as recognized by those skilled in the relevant art in order to receive such a signal and control mechanism 116. Such a signal may be transmitted by wire or wirelessly.

At step 308, user inserts needle 104 attached to device 100 into the appropriate location on the users body.

At step 310, user dispenses medication from cartridge 108. This is done by depressing the associated button 118 communicatively connected electronically or physically such as to engage dispense mechanism 116, which pushes medicine through needle 104 and into the body of the user.

At step 312, user removes needle 104 from the users body and either removes it from body 102 of device 100, retracts it into body 102 by using the appropriate button 118 or other mechanism, and/or covers it with cap 122. Cartridge 108 may be removed from body 102 as well if it is attached to needle 104 or no longer has any dosages remaining. A removed needle 104 and/or removed cartridge 108 may be disposed of or may be stored for later use.

In some aspects, memory devices within controlling electronics 110 may store data indicative of the size of the dosage of medicine that was administered, as well as the date and time of administration.

Process 300 terminates at step 314.

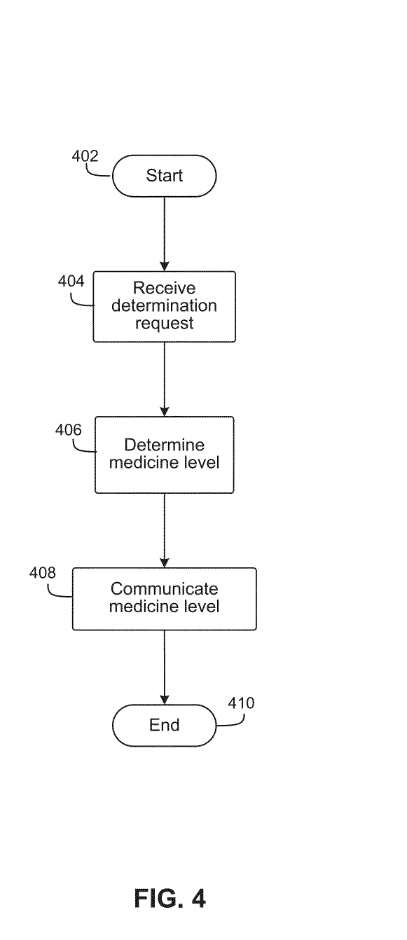

Referring now to FIG. 4, a flowchart illustrating an exemplary process 400 for facilitating the determination of how much medicine is remaining in injection device 100 for generating audio communications of medicinal dosages, according to an aspect of the present disclosure, is shown.

Process 400, which may be executing utilizing device 100, begins at step 402 with control passing immediately to step 404.

At step 404, injection device 100 receives a request from a user for a determination of the amount of medicine remaining within cartridge 108. A user enters such a request by pressing one of buttons 118 communicatively coupled to controlling electronics 110. The pressing of such a button may send a direct request to controlling electronics 110, or it may cause speaker 112 to function as a microphone as disclosed above, thus allowing the user to verbally state a command to make the desired determination. A user may also make such a determination request by entering it via display screen 202.

At step 406, controlling electronics 110 determine how much medicine is remaining in cartridge 108. In some aspects, this is accomplished via the access of memory within controlling electronics 110. Such memory may have stored therein the initial amount of medicine that cartridge 108 contained as well as the number and sizes of the dosages that have been dispensed from cartridge 108 since it was first inserted. Controlling electronics 110 may then use such information to perform calculations to determine how much medicine is remaining within cartridge 108. In some other aspects, controlling electronics 110 is communicatively coupled to sensors attached to cartridge holder 106. Such sensors may sense how much medicine is remaining inside cartridge 108 and communicate such information to controlling electronics 110.

At step 408, injection device 100 communicates the remaining medicine level to the user. This may be accomplished by providing a verbal audio communication via speaker 112, and/or by showing the medicine level on display screen 202. The medicine level on display screen 202 may be in the form of a percentage, a specified number of units, a number of similar dosage sizes remaining, by visual means such as a bar with a portion filled out representative of the amount of medicine left, or by any other similar means as recognized by those skilled in the relevant art(s) after reading the description herein.

Process 400 terminates at step 410.

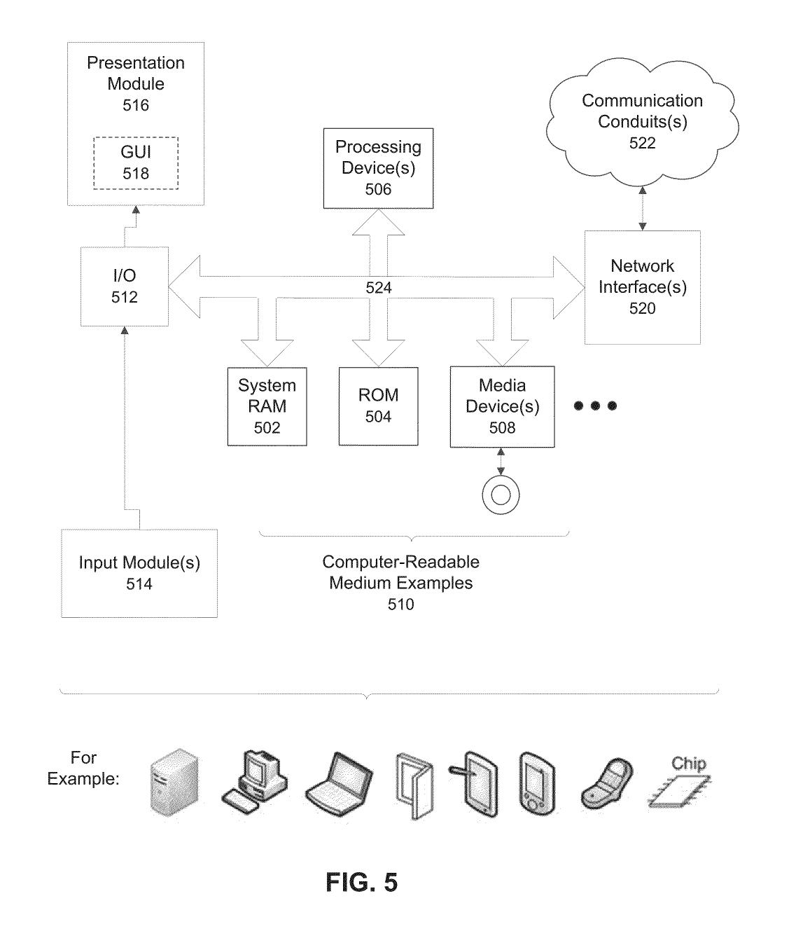

Referring now to FIG. 5, a block diagram of an exemplary computer system useful for implementing various aspects the processes disclosed herein, in accordance with one or more aspects of the present disclosure, is shown

Computing functionality 500 may comprise volatile and non-volatile memory, such as RAM 502 and ROM 504, as well as one or more processing devices 506 (e.g., one or more central processing units (CPUs), one or more graphical processing units (GPUs), and the like). Computing functionality 500 also optionally comprises various media devices 508, such as a hard disk module, an optical disk module, and so forth. Computing functionality 500 may perform various operations identified above when the processing device(s) 506 execute(s) instructions that are maintained by memory (e.g., RAM 502, ROM 504, and the like).

More generally, instructions and other information may be stored on any computer readable medium 510, including, but not limited to, static memory storage devices, magnetic storage devices, and optical storage devices. The term “computer readable medium” also encompasses plural storage devices. In all cases, computer readable medium 510 represents some form of physical and tangible entity. By way of example, and not limitation, computer readable medium 510 may comprise “computer storage media” and “communications media.”

“Computer storage media” comprises volatile and non-volatile, removable and non-removable media implemented in any method or technology for storage of information, such as computer readable instructions, data structures, program modules, or other data. Computer storage media may be, for example, and not limitation, RAM 502, ROM 504, EEPROM, Flash memory, or other memory technology, CD-ROM, digital versatile disks (DVD), or other optical storage, magnetic cassettes, magnetic tape, magnetic disk storage, or other magnetic storage devices, or any other medium which can be used to store the desired information and which can be accessed by a computer.

“Communication media” typically comprise computer readable instructions, data structures, program modules, or other data in a modulated data signal, such as carrier wave or other transport mechanism. Communication media may also comprise any information delivery media. The term “modulated data signal” means a signal that has one or more of its characteristics set or changed in such a manner as to encode information in the signal. By way of example, and not limitation, communication media comprises wired media such as a wired network or direct-wired connection, and wireless media such as acoustic, RF, infrared, and other wireless media. Combinations of any of the above are also included within the scope of computer readable medium.

Computing functionality 500 may also comprise an input/output module 512 for receiving various inputs (via input modules 514), and for providing various outputs (via one or more output modules). One particular output module mechanism may be a presentation module 516 and an associated GUI 518. Computing functionality 500 may also include one or more network interfaces 520 for exchanging data with other devices via one or more communication conduits 522. In some embodiments, one or more communication buses 524 communicatively couple the above-described components together.

Communication conduit(s) 522 may be implemented in any manner (e.g., by a local area network, a wide area network (e.g., the Internet), and the like, or any combination thereof). Communication conduit(s) 522 may include any combination of hardwired links, wireless links, routers, gateway functionality, name servers, and the like, governed by any protocol or combination of protocols.

Alternatively, or in addition, any of the functions described herein may be performed, at least in part, by one or more hardware logic components. For example, without limitation, illustrative types of hardware logic components that may be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), and the like.

The terms “module” and “component” as used herein generally represent software, firmware, hardware, or combinations thereof. In the case of a software implementation, the module or component represents program code that performs specified tasks when executed on a processor. The program code may be stored in one or more computer readable memory devices. The features of the present disclosure described herein are platform-independent, meaning that the techniques can be implemented on a variety of commercial computing platforms having a variety of processors (e.g., set-top box, desktop, laptop, notebook, tablet computer, personal digital assistant (PDA), mobile telephone, smart telephone, gaming console, and the like)

While various aspects of the present disclosure have been described above, it should be understood that they have been presented by way of example and not limitation. It will be apparent to persons skilled in the relevant art(s) that various changes in form and detail can be made therein without departing from the spirit and scope of the present disclosure. Thus, the present disclosure should not be limited by any of the above described exemplary aspects, but should be defined only in accordance with the following claims and their equivalents.

In addition, it should be understood that the figures in the attachments, which highlight the structure, methodology, functionality and advantages of the present disclosure, are presented for example purposes only. The present disclosure is sufficiently flexible and configurable, such that it may be implemented in ways other than that shown in the accompanying figures (e.g., implementation within computing instruments and environments other than those mentioned herein). As will be appreciated by those skilled in the relevant art(s) after reading the description herein, certain features from different aspects of the systems, methods and computer program products of the present disclosure may be combined to form yet new aspects of the present disclosure.

Further, the purpose of the foregoing Abstract is to enable the U.S. Patent and Trademark Office and the public generally and especially the scientists, engineers and practitioners in the relevant art(s) who are not familiar with patent or legal terms or phraseology, to determine quickly from a cursory inspection the nature and essence of this technical disclosure. The Abstract is not intended to be limiting as to the scope of the present disclosure in any way.

What is claimed is:

1. A medical injection device, comprising:
a body configured to house a needle connectively attachable to a medicine cartridge, housed within a cartridge holder, the body comprising an upper body portion, a lower body portion and an exterior body portion;
controlling electronics;
at least one button on the exterior body portion, the at least one button communicatively coupled to the controlling electronics; and
at least one speaker communicatively coupled to the controlling electronics;
wherein the controlling electronics are configured to, via the at least one speaker, provide an audio indication of a selected dosage level.

2. The device of claim 1, the controlling electronics comprising at least one microchip.

3. The device of claim 2, the controlling electronics comprising at least one memory device.

4. The device of claim 1, the controlling electronics being capable of generating verbal audio communication through the at least one speaker.

5. The device of claim 4, wherein the at least one speaker is as micro speaker.

6. The device of claim 4, the at least one speaker further being configured such as to function as a microphone;
wherein the microphone is capable of relaying verbal commands spoken by a user to the controlling electronics;
wherein the controlling electronics are capable of interpreting such commands and generating responses thereto;
wherein the microphone may be activated by one of the at least one buttons on the exterior portion of the body.

7. The device of claim 1, the body further comprising a display mechanism on the exterior portion thereof.

8. The device of claim 7, the display mechanism comprising a small LCD screen.

9. The device of claim 8, the display mechanism further comprising a touchscreen interface.

10. The device of claim 8, the display mechanism further comprising an interface comprising at least one button.

11. The device of claim 1, the body further comprising a clip affixed to the upper portion thereof;
wherein the clip is configured to removably fasten the device to a users garment.

12. The device of claim 1, one of the at least one buttons on the exterior portion of the body being configured to elicit a desired communication from the controlling electronics, the desired communication being selected from the group consisting of: a current set dosage amount and a remaining medicine amount.

13. The device of claim 1, a button on the exterior portion of the body being configured to move the needle of the device between a position completely within the device and an exposed position protruding from the lower portion of the device via a needle retraction mechanism physically or electronically connected to the button.

14. The device of claim 1, a button on the exterior portion of the body being configured to engage a dispense mechanism connected to the medicine cartridge such that a set dosage amount of medicine communicated from the controlling electronics via a communicative coupling is pushed through the needle and into a body of a user when the button is depressed.

15. The device of claim 11, the dispense mechanism comprising at least one microchip.

16. The device of claim 1, further comprising at least one sensor attached to the cartridge holder and communicatively coupled to the controlling electronics.

17. The device of claim 1, further comprising a cap to cover the lower end portion.

18. A method of injecting medication into a user's body using a medical injection device, the medical injection device comprising: a body configured to house a needle connectively attachable to a medicine cartridge, housed within a cartridge holder, such a body further comprising an upper portion, a lower portion, and an exterior portion; controlling electronics; at least one button on the exterior portion of the body and communicatively coupled to the controlling electronics; and at least one speaker communicatively coupled to the controlling electronics, the method comprising the steps of:
(a) preparing the device for use, such as by uncapping, unretracting, or attaching the needle;
(b) setting a dosage amount to be injected by such means selected from the group consisting of: pressing one of the at least one buttons communicatively coupled to the controlling electronics repeatedly until a desired dosage amount is audibly communicated in verbal form via the at least one speaker; pressing one of the at least one buttons communicatively coupled to the controlling electronics to engage the at least one speaker as a microphone in order to dictate the desired dosage amount; and using a display mechanism mounted on the exterior portion of the body of the device with a user interface to enter the desired dosage amount;
(c) inserting the needle of the device into an appropriate location on the user's body;
(d) pressing a button on the body of the device to engage a dispense mechanism that pushes the desired dosage amount of medicine in the medicine cartridge through the needle and into the user's body;
(e) removing the needle of the device from the user's body; and
(f) storing the device for later use, such as by recapping, retracting, or removing the needle.

19. A method of determining how much medicine is in a medical injection device, the medical injection device comprising: a body configured to house a needle connectively attachable to a medicine cartridge, housed within a cartridge holder, such a body further comprising an upper portion, a lower portion, and an exterior portion; controlling electronics; at least one button on the exterior portion of the body and communicatively coupled to the controlling electronics; and at least one speaker communicatively coupled to the controlling electronics, the method comprising the steps of:
(a) receiving a medicine level determination request, such receiving comprising a means selected from the group consisting of: one of the at least one buttons being depressed to trigger a command function within the controlling electronics; one of the at least one buttons being depressed to use the at least one speaker as a microphone and receiving a verbal command which is interpreted by the controlling electronics; and receiving a command via a display mechanism with a user interface mounted on the exterior portion of the body;
(b) determining an amount of medicine currently remaining in the medicine cartridge within the cartridge holder, such determining being accomplished by a means selected from the group consisting of: sensing, via at least one sensor communicatively coupled to the controlling electronics and the cartridge holder, how much medicine is left within the medicine cartridge in the cartridge holder; and accessing stored data within the controlling electronics and calculating the amount of medicine left based on such data; and
(c) communicating the amount of medicine left within the cartridge to a user via transmitting verbal audio statements through the at least one speaker and/or displaying the amount via the display mechanism.
